(19) United States
(12) Patent Application Publication
(10) Pub. No.: US 2019/0381024 A1
(43) Pub. Date: Dec. 19, 2019

(54) NICOTINAMIDE FOR LOWERING PHOSPHATE LEVELS IN HYPERPHOSPHATEMIA

(71) Applicant: MEDICE Arzneimittel Putter GmbH & Co. KG, Iserlohn (DE)

(72) Inventor: Richard AMMER, Iserlohn (DE)

(73) Assignee: MEDICE Arzneimittel Putter GmbH & Co. KG, Iserlohn (DE)

(21) Appl. No.: 15/771,420

(22) PCT Filed: Oct. 27, 2016
(86) PCT No.: PCT/EP2016/075981

§ 371 Date: Apr. 27, 2018

(30) Foreign Application Priority Data
Oct. 27, 2015 (EP) 15191608.7

(51) Int. Cl. A61K 31/455; A61K 9/00; A61P 3/12

(52) U.S. Cl. CPC A61K 31/455; A61P 3/12; A61K 9/0053; A61K 9/0019

(57) ABSTRACT

This invention relates to a pharmaceutical preparation comprising nicotinamide in combination with phosphate lowering agents (phosphate binders) for the treatment of elevated serum phosphate levels resulting particularly from kidney failure, especially in patients undergoing hemodialysis treatment, as well as to a pharmaceutical composition comprising a pharmaceutically effective amount of a combination of nicotinamide with phosphate binders together with pharmaceutically acceptable carriers.

This invention relates to a pharmaceutical preparation comprising nicotinamide in combination with at least one phosphate lowering agent (phosphate binder) for the treatment of elevated serum phosphate levels resulting particularly from chronic kidney disease (CKD), especially in patients with end stage renal disease (ESRD) undergoing dialysis/hemodialysis treatment, as well as to a pharmaceutical composition comprising a pharmaceutically effective amount of a combination of nicotinamide with at least one phosphate binder together with at least one pharmaceutically acceptable carrier.

### 1 BACKGROUND

Hyperphosphatemia, defined as super-physiological levels of phosphate, is considered an independent risk factor for CKD patients and adequate therapy is still a challenge for which ca. 50-70% of CKD patients do not meet recommended target phosphate levels.

The main consequences of hyperphosphatemia are cardiovascular complications, which are the main cause of death in patients suffering from ESRD. At local level these complications are manifested by alterations of blood vessels either by accumulation of lipids, formation of clots and occlusion of the lumen (atherosclerosis) or by thickening and calcification of the vessel walls (arteriosclerotic media calcification).

Kidney failure is the main cause of hyperphosphatemia. Chronic renal failure (CRF) is a progressive kidney disease; when the kidney has lost all its ability of clear the blood from extensive fluid volume, electrolytes, metabolic substances, the patients cannot survive and have to be referred to dialysis. Such a last condition is defined End-Stage Renal-Disease (ESRD). One of the most crucial electrolytes is phosphate.

CKD disrupts systemic calcium and phosphate homeostasis and affects the bone, gut, and parathyroid glands. This occurs because of decreased renal excretion of phosphate and diminished renal hydroxylation of 25-hydroxyvitamin D to calcitriol (1,25 dihydroxyvitamin D) [Levin, 2007]. Progressive kidney dysfunction results in hyperphosphatemia and calcitriol deficiency. These ultimately can result in hypocalcaemia. These abnormalities directly increase PTH (parathyroid hormone) levels via sensing the Calcium-Sensing Receptor (CaSR) as potent stimulus to the release of PTH. Hyperphosphatemia is also an important factor underlying hyperparathyroidism. Although the identity of the extracellular phosphate sensor is unknown, a novel phosphaturic factor, FGF23, may be regulated by phosphate and vitamin D. This may have a role in regulating parathyroid gland function in end stage renal disease (ESRD) [Saito, 2005].

Hyperphosphatemia also lowers the levels of ionized calcium and interferes with the production of 1,25-dihydroxyvitamin D, thereby resulting in increased PTH levels. Hyperphosphatemia and secondary hyperparathyroidism with abnormalities in serum phosphate and calcium levels are associated with morbidity, renal osteodystrophy, and mortality. A number of reports have delineated an increased risk of all-cause and cardiovascular mortality in patients with disorders of mineral metabolism. Although not found in all studies, the association with decreased survival primarily involves increased phosphate, calcium, calcium x phosphate product, and/or parathyroid hormone levels. These in turn are associated with accelerated atherosclerosis, arterial calcification, and an increased risk of adverse cardiovascular outcomes and death [Block, 1998; London, 2003]

Serum phosphorus exceeding 5.5 mg/dl and calcium phosphate product over 52 mg2/dl2 each correlate with an increased risk of mortality in dialysis patients [Block, 1998].

These findings have led to recent KDOQI (Kidney Disease Outcomes Quality Initiative) recommendations for a more vigorous control of serum phosphorus to between 2.5 and 5.5 mg/dl, while maintaining calcium phosphate product at less than 55 mg2/dl2 [K/DOQI clinical practice guidelines, 2003].

Because of growing concerns relating to the relationship among cardiovascular disease, vascular calcification, and abnormalities in bone and mineral metabolism, a careful process of evidence review and expert deliberation resulted in the 2003 K/DOQI guidelines on bone metabolism [K/DOQI clinical practice guidelines, 2003].

Based upon this perspective, the following is an overview of some of the general recommendations for patients undergoing maintenance dialysis (K/DOQI recommendations, 2003; KDIGO guidelines 2009)
Therapy of elevated phosphate levels (greater than 5.5 mg/dL [>1.8 mmol/L]) that is refractory to dialysis and diet can be initiated with either calcium or non-metal salt based phosphate binders.
The use of a cocktail of oral phosphate binders is encouraged, with a limit of 1.5 grams of calcium salts (making a maximum total of 2 grams of elemental calcium per day in conjunction with dietary calcium intake).
Calcium salts should be avoided in patients with sustained intact PTH levels of <150 pg/mL, or plasma calcium levels of >9.5 mg/dL (>2.37 mmol/L). Vitamin D compounds should also be avoided or terminated in patients with calcium levels greater 9.5 mg/dL (>2.37 mmol/L).
Non-calcium-based phosphate binders are preferred in patients with severe vascular or soft-tissue calcifications.
Plasma calcium levels should be maintained at the lower end of the normal range (8.4 to 9.5 mg/dL [2.1 to 2.35 mmol/L]).
The calcium-phosphate product should be kept less than 55 mg2/mL2 (<4.4 mmol2/L2) by first focusing on controlling plasma phosphate.

Further recommendations are shown in the following table 1.

TABLE 1
Recommendations according to K/DOQI practice guidelines 2003.
GFR = estimated glomerular filtration rate, a parameter for stratifying
kidney function [K/DOQI clinical practice guidelines, 2003].

| | CKD stage | | |
|---|---|---|---|
| | 3 | 4 | 5 |
| GFR (ml/min | 30-59 | 15-29 | <15 |
| per 1.73 m2) | | | |
| Serum Phosphate, | | | |
| target | | | |
| (mg/dl) | 2.7-4.6 | 2.7-4.6 | 3.5-5.5 |
| (mmol/l) | 0.87-1.49 | 0.87-1.49 | 1.13-1.78 |
| Lab test | monthly | monthly | monthly |
| Corrected total | | | |
| Calcium | | | |
| (mg/dl) | low to normal | low to normal | 8.4-9.5 |
| (mmol/l) | low to normal | low to normal | 2.10-2.37 |
| Dialyse standard | low to normal | low to normal | low to normal |
| Lab test | monthly | monthly | monthly |
| Calcium-phosphate | | | |
| product | | | |
| (mg²/dl²) | <55 | <55 | <55 |
| (mmol²/l²) | <4.4 | <4.4 | <4.4 |
| iPTH | | | |
| (pg/ml) | 35-70 | 70-110 | 150-300 |
| (pmol/l) | | | 16.5-33.0 |
| Dialyse standard | | | 2 to 5-fold |
| | | | normal |
| Lab test | 3-6 months | 3-6 months | 3-6 months |
| Dietary phosphate | 800-1000 | 800-1000 | 800-1000 |
| intake (mg/d) | | | |
| Total elemental | 2000 | 2000 | 2000 |
| calcium intake (mg/d) | | | |

Adherence to these guidelines mandates the use of a variety of different phosphate lowering agents in many patients if the central phosphate control targets are to be achieved.

The following approaches to the treatment of hyperphosphatemia by administering products with phosphate lowering activity (phosphate lowering agents) are e.g. available:
calcium based binders, i.e. calcium acetate, calcium carbonate, calcium-magnesium-salts,
aluminium based binders, i.e. aluminum chloride and aluminum-hydro-chloride,
lanthanum carbonate,
iron containing phosphate binders (iron citrate, sucroferric oxyhydroxide),

All of them are acting by physico-chemical precipitation of the agent and phosphate taken in by diet and precipitation in the gastro-intestinal tract (i.e. are classified as phosphate binders). Moreover,
sevelamer carbonate or sevelamer HCl (polymer)
   are phosphate lowering agents which act by physico-chemical absorption of phosphate taken in by diet, being absorbed by the polymer during the gastro-intestinal passage.

Due to the mode of action, pill intake with meals is essential, high dosages are required, and patient compliance is a pre-condition, but due to a high tablet burden (3 to 6 tablets or capsules per meal) it is frequently insufficient. In consequence, up to 70% of CKD patients are still in hyperphosphatemia despite treatment with the above mentioned phosphate lowering agents (Navaneethan, 2009) and do not meet above mentioned phosphate levels recommended by KDIGO and KDOQI [K/DOQI clinical practice guidelines, 2005; KIDIGO, 2009].

In contrast, others and the present inventors have shown that nicotinamide acts in a pharmacological, pharmaco-physiological mode of action by down-regulating NaPi-IIb cotransporters predominantly expressed in the small intestine.

Extracellular phosphate homeostasis is achieved by the regulation of intestinal phosphate absorption as well as by regulation of phosphate excretion via the kidneys. Current knowledge suggests three different sodium-dependent phosphate transporters (NaPi-IIa, NaPi-IIc and NaPi-IIb) as well as two type III transporters (PiT1 and PiT2) being responsible for regulation of intestinal and renal phosphate regulation (Marks, 2010). NaPi-IIb receptors are essential for the active up-take of phosphate, which contributes to ca. 50% of phosphate uptake into serum (Katai, 1999). Further details are given in Table 2.

TABLE 2
Active phosphate transporters in kidney and intestine. According to Giral, 2009;
Marks, 2010; Sabbagh, 2011; BBM = Brush Border Membrane, MEPE = matrix extracellular
phosphoglycoprotein, PFA = Phosphonoformic acid, PO4 = Phosphate, S = Segment, VDR =
Vitamin D receptor.

| | | % of PO4 flow | | Pharmacological |
|---|---|---|---|---|
| | Distribution | rate | Physiological regulators | regulators |
| NaPi-IIa | Proximal renal | ≤70% (of renal | PTH (↓) FGF23 (↓), | PFA (↓) |
| | tubule BBM (S1-S3) | reabsorption) | High PO4 (↓) | |
| NaPi-IIb | Duodenal and jejunal | ≤50% (of | Calcitriol (↑), High PO4 (↓), | Nicotinamide (↓) |
| | BBM | intestinal | Low PO4 (↑), FGF23 (indirect ↓) | PFA (↓) |
| | | absorption) | | |
| NaPi-IIc | Proximal renal | ≥30% (of renal | FGF23 (↓), dietary PO4 (↑), | PFA (↓) |
| | tubule BBM (S1) | reabsorption) | High dietary Mg²⁺ (↑) | |
| NaPi-III | Duodenal and jejunal | No data | FGF23 (↓), High dietary PO4 (↓), | No data |
| PiT1 | BBM | | Metabolic acidosis (↑) | |
| NaPi-III | Proximal renal tubule | 3-40% | FGF23 (↓), high dietary PO4 (↓), | No data |
| PiT2 | BBM | | Metabolic acidosis (↑) | |

Inhibition of renal NaPi-IIa and NaPi-IIc protein expression either in double knockout mice (Marks, 2010) or via FGF23 (Gattineni, 2009) induce severe hypophosphatemia by blockade of tubular phosphate reabsorption in the kidneys.

Sodium dependent phosphate transporter NaPi-IIb was shown to be responsible for around 50% of gastrointestinal phosphate absorption (Katai, 1999). Beneath this transcellular transport mechanism, passive phosphate diffusion is also important in intestinal phosphate uptake. The expression of intestinal NaPi-IIb is blocked by a phosphate-rich diet (Hattenhauer, 1999). A low-phosphate diet (Giral, 2009; Hattenhauer, 1999) or an increase in serum calcitriol (Xu, 2002) increases the expression of the transporter. FGF23 was shown to exert an indirect inhibitory action on intestinal NaPi-IIb expression via inhibition of renal 1α-hydroxylase activity and therefore decreasing Calcitriol levels (Marks, 2010).

A decrease in the absorption of phosphate from the small intestine, due to inhibition of the phosphate co-transporter NaPi-IIb, can be regarded as a new mechanism of action in the reduction of phosphate concentrations. Intraperitoneal administration of nicotinamide blocks the expression of NaPi-IIb (Eto, 2005) and inhibits the gastrointestinal absorption of phosphate (Katai, 1999). It has not been established whether the functional transporter is also directly inhibited.

It has been shown that nicotinamide can be effective in lowering elevated phosphate levels in animals (Eto, 2005) and in human, as e.g. also shown in Table 3:

TABLE 3
Overview of nicotinamide studies in CKD patients

| | | | Average | | |
|---|---|---|---|---|---|
| | | | dose | Dose | treatment |
| Source | n | Patients | (mg/d) | Range | duration |
| Takahashi et al. | 65 | Hemodialysis | 1080 | 500-1750 | 12 |
| Rahmouni et al. | 10 | CKD 4-5 | 720 | 500-1000 | 9 |
| Cheng et al. 2008 | 33 | Hemodialysis | 1500 | 500-1500 | 8 |
| Young et al. 2009 | 8 | Peritonealdial | 1000 | 500-1500 | 8 |

### DESCRIPTION OF INVENTION

It is a problem of the invention to provide a new composition for the treatment of hyperphosphatemia, particularly resulting from chronic kidney failure (CKD).

This problem is solved by a pharmaceutical preparation comprising a pharmaceutically effective amount of a combination of nicotinamide with one or more phosphate binders, optionally together with pharmaceutically acceptable carriers, e.g. at least one pharmaceutically acceptable carrier or more than one pharmaceutically acceptable carrier.

The present invention also involves the administration of a pharmaceutically effective amount of nicotinamide in combination with phosphate binders for the treatment of hyperphosphatemia, resulting particularly from chronic kidney failure (CKD).

According to a further embodiment, the invention also concerns a pharmaceutical preparation comprising a pharmaceutically effective amount of nicotinamide in combination with one or more phosphate binders in a method for the treatment of hyperphosphatemia, resulting particularly from chronic kidney (renal) diseases, as well as for the treatment of End-Stage Renal Disease (ESRD).

The pharmaceutical preparation is administered preferably via the oral route or the parenteral route.

This invention involves the administration of a pharmaceutically effective quantity of nicotinamide in combination with one or more phosphate binders, particularly known phosphate lowering agents, such as
calcium based binders, i.e. calcium acetate, calcium carbonate, calcium-magnesium-salts, and/or
aluminum based binders, i.e. aluminum chloride and aluminum-hydrochloride, and/or
lanthanum carbonate, and/or
iron containing phosphate binders (iron citrate, sucroferric oxyhydroxide), and/or
sevelamer carbonate or sevelamer HCl (polymers).

The phosphate binders of the invention are also named phosphate lowering agents and are known in the art per se. According to the invention, also other phosphate binders acting in lowering the phosphate level can be used within the scope of the invention. The terms “phosphate lowering agents” and “phosphate binders” are used herein, within the scope of the invention, interchangeably.

Further embodiments and advantages of the invention can be taken form the following description, figures, the examples as well as the dependent claims, without being limited thereto.

### FIGURES

The enclosed drawings should illustrate embodiments of the present invention and convey a further understanding thereof. In connection with the description they serve as explanation of concepts and principles of the invention. Other embodiments and many of the stated advantages can be derived in relation to the drawings. The elements of the drawings are not necessarily to scale towards each other. Identical, functionally equivalent and acting equal features and components are denoted in the figures of the drawings with the same reference numbers, unless noted otherwise.

FIG. 1 shows the level of expression for NaPi-IIb receptor in the small intestine in the present Example.

FIG. 2 shows phosphate levels (mmol/l) in CKD patients on hemodialysis in response to known phosphate lowering agents over time.

FIG. 3 depicts the Phosphate levels (mmol/l) in CKD patients on hemodialysis in response to nicotinamide alone over time.

FIG. 4 shows the phosphate levels (mmol/l) in CKD patients on hemodialysis in response to known phosphate lowering agents in combination with nicotinamide over time.

FIG. 5 shows a dose-effect curve for phosphate levels (mmol/l) in response to nicotinamide administered in CKD patients in hyperphosphatemia.

### DEFINITIONS

Unless defined otherwise, technical and scientific terms used herein have the same meaning as commonly understood by one of ordinary skill in the art to which this invention belongs. The numerical figures provided herein like the unit doses of nicotinamide have to be understood as covering also “about” values.

In a first aspect the present invention relates to a pharmaceutical preparation comprising a pharmaceutically effective amount of a combination of nicotinamide with at least one phosphate binder, e.g. also phosphate binders, i.e. more than one phosphate binder, for use in a method of treating of elevated serum phosphate levels (hyperphosphatemia), particularly resulting from renal failure, as well as for the treatment of End-Stage Renal Disease (ESRD).

In this regard elevated phosphate levels are phosphate levels which exceed those recommended by medical guidelines, e.g. serum phosphate levels exceeding about 5.5 mg/dl and/or with serum phosphate levels about 1.78 mmol/l.

The phosphate binder is not particularly restricted in this regard and those usually applied for the treatment of hyperphosphatemia can be applied. According to certain embodiments the phosphate binder is at least one selected from the group comprising
calcium based binders, e.g. calcium acetate, calcium carbonate, calcium-magnesium-salts,
aluminum based binders, e.g. aluminum chloride and aluminum-hydrochloride,
lanthanum carbonate,
iron containing phosphate binders, e.g. iron citrate, sucroferric oxyhydroxide, and/or
sevelamer carbonate or sevelamer HCl (polymers).

Also further constituents of the pharmaceutical preparation for use in a method of treating of elevated serum phosphate levels like at least one pharmaceutically acceptable carrier and/or other excipients like antiadherents, binders, coatings, colors, disintegrants, flavors, fillers, diluents, glidants, lubricants, preservatives, sorbents, sweeteners and/or vehicles are not particularly restricted.

Described is also a method of treating elevated serum phosphate levels (hyperphosphatemia), particularly resulting from renal failure, as well as for the treatment of End-Stage Renal Disease (ESRD) comprising administration of a pharmaceutically effective amount of a combination of nicotinamide with phosphate binders, particularly at least one phosphate binder. Certain embodiments of such a method are also explained with regard to the pharmaceutical preparation of the first aspect of the present invention.

According to certain embodiments, the hyperphosphatemia results from chronic kidney failure, from of end-stage renal disease, and/or from hemodialysis.

According to certain embodiments, the pharmaceutical preparation of the first aspect is administered parenterally or orally.

According to certain embodiments, the nicotinamide in the pharmaceutical preparation of the first aspect is administered in unit doses ranging from about 250 to 2000 mg, preferably about 250 to 1000 mg, per day. According to certain embodiments, the nicotinamide is to be administered before, with and/or after meals, independently from food intake and before and/or after hemodialysis or peritoneal dialysis treatment.

According to certain embodiments, the phosphate binder(s) can be administered in unit doses which are known in the art and can be adjusted by the skilled person with respect to the disease and the individual patient to be treated as well as in relationship to the amount of the nicotinamide used and the kind of phosphate binder selected.

Usual unit doses vary according to phosphate binder applied, while, at least for some patients, the recommended daily dose (KDIGO 2009, DIMDI and WHO ATC defined daily doses) is as follows,
calcium based binders, e.g. calcium acetate (about 5600-6300 mg/d, e.g. ca. 6000 mg/d), calcium carbonate (ca. 4000 mg/d), calcium-magnesium-salts (about 4000-4500 mg/d, e.g. ca-4226 mg/d), not exceeding the recommended daily unit dose of ca. 1500 mg elementary calcium per day
aluminum-based binders, e.g. aluminum chloride, Al₉Cl₈(OH)₁₉ (about 900-1800 mg/d) and aluminum hydrochloride (about 1800-12000 mg/d), daily dose is e.g. ca. 1800 mg/d
lanthanum carbonate, daily dose is e.g. about 3708 mg/d, and/or average daily dose is e.g. about 2250 mg/d
iron containing phosphate binders, e.g. iron citrate, sucroferric oxyhydroxide, daily dose is ca. 7200-7500 mg/d
sevelamer carbonate or sevelamer HCl (polymers), daily dose is ca. 5600-6400 mg/d.

The above recited doses may vary as written above and can be adjusted by the skilled person with respect to the disease and the individual patient to be treated as well as in relationship to the amount of the nicotinamide used and the kind of phosphate binder selected.

According to certain embodiments, the pharmaceutical preparation is in the form of tablets, capsules, oral preparations, powders, granules, lozenges, reconstitutable powders, syrups, solutions or suspensions

In a further aspect, the present invention relates to a pharmaceutical preparation comprising a pharmaceutically effective amount of a combination of nicotinamide with at least one phosphate binder together with at least one pharmaceutically acceptable carrier.

The phosphate binder can thereby be the same as described with regard to the first aspect of the present invention, i.e. the pharmaceutical preparation comprising a pharmaceutically effective amount of a combination of nicotinamide with at least one phosphate binder, e.g. also phosphate binders, i.e. more than one phosphate binder, for use in a method of treating of elevated serum phosphate levels. Again, more than one phosphate binder, i.e. two or more phosphate binders, can be used.

The at least one pharmaceutically acceptable carrier is not particularly limited as long as it is compatible with the nicotinamide and/or the at least one phosphate binder and can be suitably selected based on the type of phosphate binder, dosage form, etc., and can e.g. also be adjusted to special patient needs. More than one pharmaceutically acceptable carrier can be used in the pharmaceutical preparation, i.e. two or more pharmaceutically acceptable carriers.

Regarding the dosage of the at least one phosphate binder and/or nicotinamide in a dosage form, reference can also be made to the established principles of pharmacology in human and veterinary medicine. For example, Forth, Henschler, Rummel “Allgemeine und spezielle Pharmakologie und Toxikologie”, 9th edition, 2005, pp. 781-919, might be used as a guideline. Regarding the formulation of a ready-to-use medicament, reference can made to “Remington, The Science and Practice of Pharmacy”, 22^{nd} edition, 2013, pp. 777-1070. The contents thereof are incorporated by reference.

In this regard the pharmaceutical preparation can be also provided in a form with more than one dosage form, e.g. in the form of a kit-of-parts. In such a kit-of-parts one dosage form can e.g. comprise nicotinamide and a further one can comprise at least one phosphate binder. The two or more dosage forms in the kit-of-parts can then each comprise at least one pharmaceutically acceptable carrier which can be the same or different. According to certain embodiments the pharmaceutical preparation can be a dosage form comprising both nicotinamide and at least one phosphate binder together with at least one pharmaceutically acceptable carrier.

The pharmaceutical preparation can comprise further pharmaceutically acceptable excipients like antiadherents, binders, coatings, colours, disintegrants, flavors, fillers, diluents, glidants, lubricants, preservatives, sorbents, sweeteners, water stabilizers, antifungals and/or vehicles which preferably do not interact with the nicotinamide and/or at least one phosphate binder.

These excipients are well-known to the skilled person, e.g. from Remington, The Science and Practice of Pharmacy, 22nd Edition, 2012, which is incorporated herein by reference in regard to pharmaceutical excipients, particularly volume 1: “The Science of Pharmacy”, pages 1049-1070 or from Rowe, R. C., Sheskey, P. J., Quinn, M. E., Cook, W. G., Fenton, M. E., “Handbook of Pharmaceutical Excipients”, 7th Edition, 2012, which is incorporated herein by reference in regard to pharmaceutical excipients.

The pharmaceutical preparation can be used for treating elevated serum phosphate levels (hyperphosphatemia), particularly resulting from renal failure, particularly wherein said hyperphosphatemia results from chronic kidney failure, from of end-stage renal disease, and/or from hemodialysis. It can be administered parenterally and/or orally, e.g. one dosage form can be administered parenterally and one orally in case of a kit-of-parts.

The pharmaceutical preparation can be administered in unit doses ranging from 250 to 2000 mg per day, and can be administered before, with and/or after meals, independently from food intake and before and/or after hemodialysis or peritoneal dialysis treatment.

According to certain embodiments, the pharmaceutical preparation is in the form of tablets, capsules, oral preparations, powders, granules, lozenges, reconstitutable powders, syrups, solutions or suspensions

The above embodiments can be combined arbitrarily, if appropriate. Further possible embodiments and implementations of the invention comprise also combinations of features not explicitly mentioned in the foregoing or in the following with regard to the Examples of the invention. Particularly, a person skilled in the art will also add individual aspects as improvements or additions to the respective basic form of the invention.

### Examples

The present invention will now be described in detail with reference to several examples thereof. However, these examples are illustrative and do not limit the scope of the invention.

A study has been undertaken wherein administration of a conventional phosphate binder as well as administration of nicotinamide has been compared with the administration of a combination of a phosphate binder and nicotinamide. For concrete numbers of the amount of patients tested as well as with regard to the phosphate binder and the dosing of phosphate binder and nicotinamide, further details can be taken from the following description, also with regard to the Figures.

It has been found out that a combination of nicotinamide with at least one phosphate binder shows surprising advantages over a treatment with nicotinamide or at least one phosphate binder alone, as detailed in the following.

The invention is remarkable as one would expect treatment with known phosphate lowering agents acting by precipitation or absorption is sufficient for reducing uptake of phosphate. However, we have shown that known phosphate lowering agents with physico-chemical mode of action lead to an increased expression of NaPi-IIb in the small intestine which enhances the active uptake of phosphate into the serum, i.e. is contra-productive to the therapeutic approach intended. Thus, down-regulation and reducing the extent of phosphate transporter NaPi-IIb being expressed contributes heavily to the effective management of hyperphosphatemia.

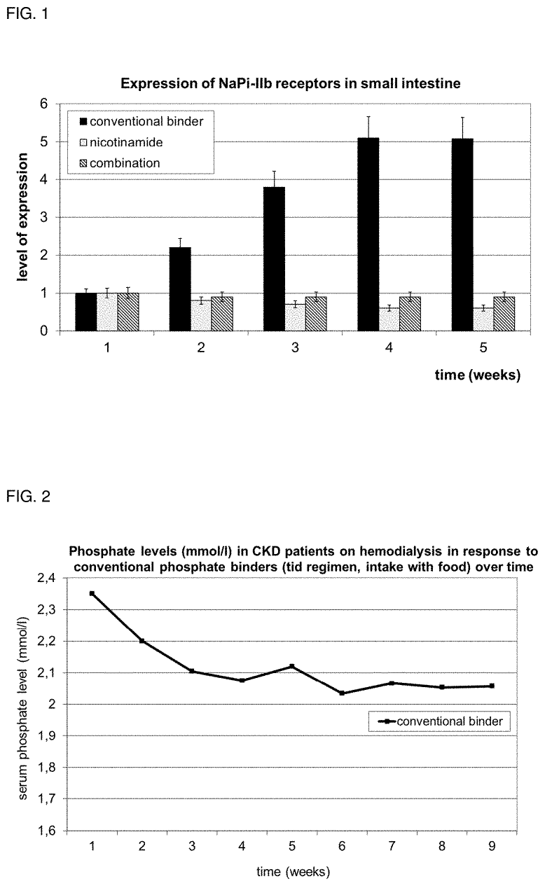

The expression of NaPi-IIb receptors in small intestine for different treatments is shown in FIG. 1. FIG. 1 shows the level of expression for NaPi-IIb receptor in the small intestine over the course of 5 weeks, either being treated with known phosphate lowering agents (conventional binder, i.e. calcium carbonate 10 mg/kg body weight, sevelamer 20 mg/kg body weight), or nicotinamide (dose 10 mg/kg body weight) or a combination of both medications (data on file).

In consequence, as seen from FIG. 1, the combination of known phosphate lowering agents with nicotinamide complementarily and synergistically enforces the therapeutic approach aiming for reducing elevated phosphate levels in serum:
complementarily, as known phosphate lowering agents lead to a decreased supply of phosphate available for active and passive uptake, but trigger increased NaPi-IIb expression enhancing active phosphate uptake. Nicotinamide, in contrast, downregulates the expression of NaPi-IIb receptors and thus, limits the active uptake of phosphate from the gut into the serum.
synergistically, as we have been able to show that the combination of nicotinamide and known phosphate lowering agents leads to a more profound and superior reduction of serum phosphate in comparison to the dose-equivalent alternative approach alone, i.e. with nicotinamide alone or with known phosphate lowering agents alone.

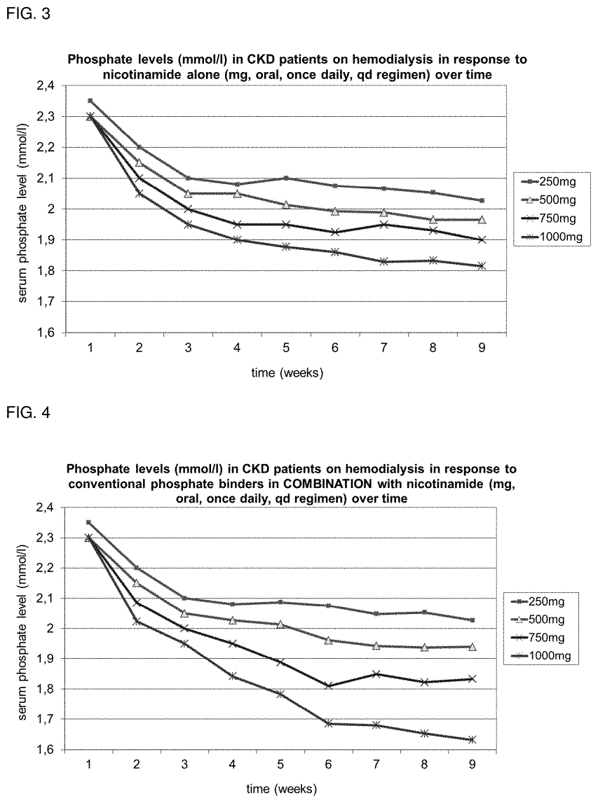

These findings are derived from several clinical findings in which chronic renal insufficient patients underwent various medication regimes after an initial wash out phase over 4 weeks to eliminate and to control for pre-medication effects. Patients were either put on their previous drug regimen consisting of various known phosphate lowering agents taken 3-times a day simultaneously to regular meals (FIG. 2), resulting in 38% of patients with serum phosphate levels <1.78 mmol/l, or
switched to various dosages of nicotinamide alone (FIG. 3), which achieved phosphate levels <1.78 mmol/l in 45% of patients, or
switched to various dosages of nicotinamide as add on medication in combination with previous conventional phosphate binder therapy (FIG. 4), which resulted in 69% of patients achieving target levels <1.78 mmol/l.

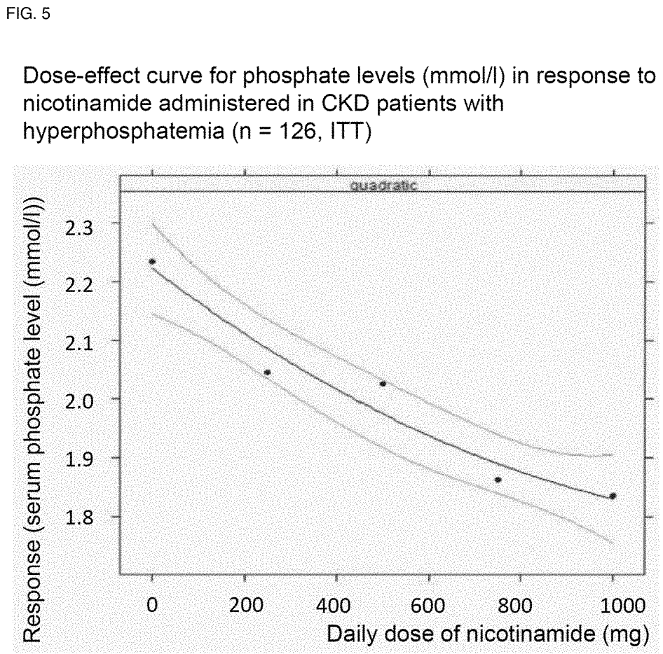

A dose-response association was drawn and calculated with confidence intervals for nicotinamide therapy (FIG. 5).

FIG. 2 thereby shows phosphate levels (mmol/l) in CKD patients on hemodialysis in response to known phosphate lowering agents (tid regimen, intake with food) over time. It shows a dose-effect curve for phosphate levels (mmol/l) in response to therapy with known phosphate lowering agents alone (calcium based, sevelamer, lanthan) administered in CKD patients (n=252, ITT=intent-to treat) over a treatment course of 9 weeks.

FIG. 3 shows phosphate levels (mmol/l) in CKD patients on hemodialysis in response to nicotinamide alone (mg, oral, once daily, qd regimen) over time. It shows a dose-effect curve for phosphate levels (mmol/l) in response to nicotinamide alone (doses 250 to 1000 mg) administered in CKD patients (n=126, ITT) over a treatment course of 9 weeks.

FIG. 4 shows phosphate levels (mmol/l) in CKD patients on hemodialysis in response to known phosphate lowering agents in COMBINATION with nicotinamide (mg, oral, once daily, qd regimen) over time. It shows a dose-effect curve for phosphate levels (mmol/l) in response to add on nicotinamide (doses 250 to 1000 mg) in combination to therapy with known phosphate lowering agents administered in CKD patients (n=126, ITT) over a treatment course of 9 weeks.

FIG. 5 shows a dose-effect curve for phosphate levels (mmol/l) in response to nicotinamide administered in CKD patients (n=126, ITT) in hyperphosphatemia.

In conclusion, according to our findings, nicotinamide in unit daily doses of 250 to 2,000 mg is effective in lowering elevated phosphate levels in combination with known phosphate lowering agents and can achieve target levels of normalized serum phosphate in up to 50 to 70% of CKD patients, whereas known phosphate lowering agents alone achieved normalization only in ca. 30% of CKD patients. Whereas known phosphate lowering agents can only work when taken with food intake, nicotinamide seems to have its beneficial effect independently from food intake, even if taken just once a day (q.d. regimen).

According to the invention, the pharmaceutical composition (preparation) comprising nicotinamide and phosphate lowering agents may be in the form of tablets, capsules, oral preparations, powders, granules, lozenges, reconstitutable powders, syrups, solutions or suspensions. The pharmaceutical composition (preparation) may also contain pharmaceutically compatible excipients and carriers known in the art per se.

### LITERATURE

Block G A, Hulbert-Shearon T E, Levin N W, et al. Association of serum phosphorus and calcium x phosphate product with mortality risk in chronic hemodialysis patients: a national study. Am J Kidney Dis 1998; 31: 607-617.
Cheng, S. C., D. O. Young, Y. Huang, J. A. Delmez, and D. W. Coyne, 2008, A Randomized, Double-Blind, Placebo-Controlled Trial of Niacinamide for Reduction of Phosphorus in Hemodialysis Patients: Clin. J Am Soc Nephrol., v. 4, no. 3, p. 1131-1138.
Goodman, W G, Goldin, J, Kuizon, B D, et al. Coronary-artery calcification in young adults with end-stage renal disease who are undergoing dialysis. N Engl J Med 2000; 342:1478
Eto, N., M. Tomita, and M. Hayashi, 2006, NaPi-mediated transcellular permeation is the dominant route in intestinal inorganic phosphate absorption in rats: Drug Metab Pharmacokinet., v. 21, no. 3, p. 217-221
Giral, H. et al., 2009, Regulation of rat intestinal Na-dependent phosphate transporters by dietary phosphate: Am. J. Physiol Renal Physiol, v. 297, no. 5, p. F1466-F1475
Hattenhauer, O., M. Traebert, H. Murer, and J. Biber, 1999, Regulation of small intestinal Na-P(i) type IIb cotransporter by dietary phosphate intake: Am. J. Physiol, v. 277, no. 4 Pt 1, p. G756-G762.
Katai, K. et al., 1999, Nicotinamide inhibits sodium-dependent phosphate cotransport activity in rat small intestine: Nephrol Dial Transplant, v. 14, no. 5, p. 1195-1201.
KDIGO. Clinical Practice Guideline for the Diagnosis, Evaluation, Prevention, and Treatment of Chronic Kidney Disease-Mineral and Bone Disorder (CKD-MBD). Kidney International 76[Supplement 113], S1-S130. 2009.
K/DOQI Clinical Practice Guidelines for Bone Metabolism and Disease in Chronic Kidney Disease. Am J Kidney Dis 2003; 42(Suppl 3):S1-201, accessible via the National Kidney Foundation's web site at https://www.kidney.org/sites/default/files/docs/boneguidelines.pdf.
Levin, A, Bakris, G L, Molitch, M, et al. Prevalence of abnormal serum vitamin D, PTH, calcium, and phosphorus in patients with chronic kidney disease: Results of the study to evaluate early kidney disease. Kidney Int 2007; 71:31.
London, G M, Guerin, A P, Marchais, S J, et al. Arterial media calcification in end-stage renal disease: impact on all-cause and cardiovascular mortality. Nephrol Dial Transplant 2003; 18:1731
Marks, J., E. S. Debnam, and R. J. Unwin, 2010, Phosphate homeostasis and the renal gastrointestinal axis: American Journal of Physiology—Renal Physiology, v. 299, no. 2, p. F285-F296
National Kidney Foundation, 2005, Clinical practice guidelines for bone metabolism and disease in chronic kidney disease, Am J Kidney Dis,
Navaneethan, S. D., S. C. Palmer, J. C. Craig, G. J. Elder, and G. F. Strippoli, Benefits and harms of phosphate binders in CKD: a systematic review of randomized controlled trials: Am J Kidney Dis. 2009; 54:4, 619-637
Rahmouni, K, B Araar, L Harbouche, I Shahapuni, N E Esper, A Fournier. Use of nicotinamide as phosphate binder could reduce by half the cost of hyperphosphatemia control by Sevelamer in dialysis patients. SP240. 2005.
Saito, H, Maeda, A, Ohtomo, S, et al. Circulating FGF-23 is regulated by 1alpha,25 dihydroxyvitamin D3 and phosphorus in vivo. J Biol Chem 2005; 280:2543.
Takahashi, Y., A. Tanaka, T. Nakamura, T. Fukuwatari, K. Shibata, N. Shimada, I. Ebihara, and H. Koide, 2004, Nicotinamide suppresses hyperphosphatemia in hemodialysis patients: Kidney Int., v. 65, no. 3, p. 1099-1104.
Young, D. O., S. C. Cheng, J. A. Delmez, and D. W. Coyne, 2009, The effect of oral niacinamide on plasma phosphorus levels in peritoneal dialysis patients: Perit. Dial. Int., v. 29, no. 5, p. 562-567.

1. A pharmaceutical preparation comprising a pharmaceutically effective amount of a combination of nicotinamide with at least one phosphate binder and optionally pharmaceutically acceptable carriers for use in a method of treating of elevated serum phosphate levels (hyperphosphatemia), particularly resulting from renal failure.

2. The pharmaceutical preparation of claim 1, wherein said hyperphosphatemia results from chronic kidney failure, from of end-stage renal disease, and/or from hemodialysis.

3. The pharmaceutical preparation of claim 1, wherein it is administered parenterally or orally.

4. The pharmaceutical preparation of claim 1, wherein the nicotinamide is to be administered in unit doses ranging from about 250 to 2000 mg per day.

5. The pharmaceutical preparation of claim 1, wherein the nicotinamide is to be administered before, with and/or after meals, independently from food intake and before and/or after hemodialysis or peritoneal dialysis treatment.

6. A pharmaceutical preparation comprising a pharmaceutically effective amount of a combination of nicotinamide with at least one phosphate binder together with at least one pharmaceutically acceptable carrier.

7. A method of treating elevated serum phosphate levels (hyperphosphatemia) in a subject, comprising administering to the subject a pharmaceutical preparation comprising a pharmaceutically effective amount of a combination of nicotinamide with at least one phosphate binder and optionally pharmaceutically acceptable carriers.

8. The method of claim 7, wherein the subject has hyperphosphatemia as a result of renal failure.

9. The method of claim 7, wherein the subject has hyperphosphatemia as a result of chronic kidney failure, from of end-stage renal disease, and/or from hemodialysis.

10. The method of claim 7, wherein the pharmaceutical preparation is administered parenterally or orally.

11. The method of claim 7, wherein the nicotinamide is administered in unit doses ranging from about 250 to 2000 mg per day.

12. The method of claim 7, wherein the nicotinamide is administered before, with and/or after meals, independently from food intake and before and/or after hemodialysis or peritoneal dialysis treatment.
